(19) United States
(12) Patent Application Publication
(10) Pub. No.: US 2025/0288635 A1
(43) Pub. Date: Sep. 18, 2025

(54) OVER-THE-COUNTER TRADITIONAL CHINESE MEDICINE FOR WEIGHT LOSS

(71) Applicant: Chongqing Kangershou Biopharmaceutical Technology Co., Ltd, Chongqing (CN)

(72) Inventor: Shucai Deng, Chongqing (CN)

(21) Appl. No.: 18/639,308

(22) Filed: Apr. 18, 2024

(30) Foreign Application Priority Data
Mar. 16, 2024 (CN) 2024103015055

(51) Int. Cl. A61K 36/481; A61K 35/38; A61K 36/074; A61K 36/233; A61K 36/258; A61K 36/287; A61K 36/42; A61K 36/488; A61K 36/708; A61K 36/73; A61K 36/744; A61K 36/81; A61K 36/8998; A61P 3/04

(52) U.S. Cl. CPC A61K 36/481; A61K 35/38; A61K 36/074; A61K 36/233; A61K 36/258; A61K 36/287; A61K 36/42; A61K 36/488; A61K 36/708; A61K 36/73; A61K 36/744; A61K 36/81; A61K 36/8998; A61P 3/04

(57) ABSTRACT

An over-the-counter traditional Chinese medicine for weight loss comprises the following raw materials in parts by weight: 30˜60 g of stir-fried yellow soybeans, 25˜50 g of Gynostemma pentaphyllum, 10˜15 g of Radix et Rhizoma ginseng, 8˜10 g of Radix Astragali, 8˜16 g of Fructus Lycii, 8˜16 g of Radix Puerariae Lobatae, 6˜12 of Radix Bupleuri, 6˜12 g of Fructus Crataegi, 6˜12 g of Semen Cassiae, 6˜12 g of Fructus Gardeniae, 6˜12 g of Fructus Hordei Germinatus, 6˜12 g of Folium Nelumbinis, 4˜8 g of Rhizoma Polygonati, 8˜10 g of Endothelium Corneum Gigeriae Galli, 4˜8 g of Flos Chrysanthemi, 4˜8 g of Radix Angelicae Sinensis, 3˜6 g of Radix et Rhizoma Rhei, 3˜6 g of Ganoderma. The advantages of the invention are as follows: reasonable design of raw materials, convenient production, processing using modern biological cell wall-breaking technology; ensuring that effective components of traditional Chinese medicine, especially bioactive enzymes, remain intact; it has good weight loss effects, is healthy and reliable, has excellent promotion prospects, economic value.

### TECHNICAL FIELD

The invention relates to the technical field of weight loss, in particular to an over-the-counter traditional Chinese medicine for weight loss.

### BACKGROUND ART

Complications caused by obesity are numerous, including metabolic syndrome, cardiovascular disease, hypertension, atherosclerosis, coronary heart disease, type 2 diabetes, osteoarthritis, cancer, etc. The harm of obesity can affect almost all organ systems in the body and is closely associated with an increased risk of sudden death.

How to effectively solve the difficulty, poor effect and high cost of losing weight has become a research focus in the field of weight loss.

### SUMMARY OF THE INVENTION

The object of the invention is to provide an over-the-counter traditional Chinese medicine for weight loss to address the problems raised in the above background.

To solve the above technical problems, the invention provides the following technical scheme: an over-the-counter traditional Chinese medicine for weight loss comprises the following raw materials in parts by weight: 30˜60 g of stir-fried yellow soybeans, 25˜50 g of Gynostemma pentaphyllum, 10˜15 g of Radix et Rhizoma ginseng, 8˜10 g of Radix Astragali, 8˜16 g of Fructus Lycii, 8˜16 g of Radix Puerariae Lobatae, and 6˜12 g of Radix Bupleuri, 6˜12 g of Fructus Crataegi, 6˜12 g of Semen Cassiae, 6˜12 g of Fructus Gardeniae, 6˜12 g of Fructus Hordei Germinatus, 6˜12 g of Folium Nelumbinis, 4˜8 g of Rhizoma Polygonati, 8˜10 g of Endothelium Corneum Gigeriae Galli, 4˜8 g of Flos Chrysanthemi, 4˜8 g of Radix Angelicae Sinensis, 3˜6 g of Radix et Rhizoma Rhei, 3˜6 g of Ganoderma.

Preferably, the over-the-counter traditional Chinese medicine for weight loss comprises the following raw materials in parts by weight: 30 g of stir-fried yellow soybeans, 50 g of Gynostemma pentaphyllum, 10 g of Radix et Rhizoma ginseng, 10 g of Radix Astragali, 8 g of Fructus Lycii, 16 g of Radix Puerariae Lobatae, 6 g of Radix Bupleuri, 12 g of Fructus Crataegi, 6 g of Semen Cassiae, 12 g of Fructus Gardeniae, 6 g of Fructus Hordei Germinatus, 12 g of Folium Nelumbinis, 4 g of Rhizoma Polygonati, 10 g of Endothelium Corneum Gigeriae Galli, 4 g of Flos Chrysanthemi, 8 g of Radix Angelicae Sinensis, 3 g of Radix et Rhizoma Rhei, 6 g of Ganoderma.

Preferably, the over-the-counter traditional Chinese medicine for weight loss comprises the following raw materials in parts by weight: 60 g of stir-fried yellow soybeans, 25 g of Gynostemma pentaphyllum, 15 g of Radix et Rhizoma ginseng, 8 g of Radix Astragali, 16 g of Fructus Lycii, 8 g of Radix Puerariae Lobatae, 12 g of Radix Bupleuri, 6 g of Fructus Crataegi, 12 g of Semen Cassiae, 6 g of Fructus Gardeniae, 12 g of Fructus Hordei Germinatus, 6 g of Folium Nelumbinis, 8 g of Rhizoma Polygonati, 10 g of Endothelium Corneum Gigeriae Galli, 8 g of Flos Chrysanthemi, 4 g of Radix Angelicae Sinensis, 6 g of Radix et Rhizoma Rhei, 3 g of Ganoderma.

Preferably, the over-the-counter traditional Chinese medicine for weight loss comprises the following raw materials in parts by weight: 45 g of stir-fried yellow soybeans, 38 g of Gynostemma pentaphyllum, 13 g of Radix et Rhizoma ginseng, 9 g of Radix Astragali, 12 g of Fructus Lycii, 12 g of Radix Puerariae Lobatae, 9 g of Radix Bupleuri, 9 g of Fructus Crataegi, 9 g of Semen Cassiae, 9 g of Fructus Gardeniae, 9 g of Fructus Hordei Germinatus, 9 g of Folium Nelumbinis, 6 g of Rhizoma Polygonati, 9 g of Endothelium Corneum Gigeriae Galli, 6 g of Flos Chrysanthemi, 6 g of Radix Angelicae Sinensis, 5 g of Radix et Rhizoma Rhei, 4 g of Ganoderma.

Dosage and Administration: taking two small bags daily, each bag containing 6 grams, drinking one bag in the morning and one at noon, brewing it in boiling water, consuming it before or after meals (better effect if taken before meals), finishing drinking the entire dosage.

The advantages of the invention are as follows: reasonable design of raw materials, convenient production, processing using modern biological cell wall-breaking technology; ensuring that the effective components of traditional Chinese medicine, especially bioactive enzymes, remain intact; it has good weight loss effects, is healthy and reliable, and has excellent promotion prospects and economic value.

### SPECIFIC EMBODIMENT OF THE INVENTION

For purposes of the following detailed description, it should be understood that the invention is capable of various alternative variations and step sequences unless expressly stated to the contrary. Furthermore, except in any operating embodiments, or where otherwise indicated, all numbers expressing, for example, amounts of ingredients used in the specification and claims are to be understood as modified in all instances by the term “about”. Therefore, unless indicated to the contrary, the numerical parameters set forth in the following specification and appended claims are approximations that will vary depending upon the desired properties to be obtained with the invention. At the very least, and not as an attempt to limit the application of the doctrine of equivalents to the scope of the claims, each numerical parameter should at least be explained based on the number of reported significant digits and rounded using common rounding techniques.

Although the numerical ranges and parameters describing the wide scope of the invention are approximate values, the specific values listed in embodiments should be reported as precisely as possible. However, any numerical value inherently includes some error arising from the standard deviation found in its respective tests and measurements.

Furthermore, it should be understood that any numerical range stated herein is intended to include all subranges subsumed therein. For example, the range “1 to 10” is intended to include all subranges between (and including) the minimum value 1 and the maximum value 10, i.e., having a minimum value equal to or greater than 1 and a maximum value equal to or less than 10.

### Embodiment 1

The over-the-counter traditional Chinese medicine for weight loss comprises the following raw materials in parts by weight: 30 g of stir-fried yellow soybeans, 50 g of Gynostemma pentaphyllum, 10 g of Radix et Rhizoma ginseng, 10 g of Radix Astragali, 8 g of Fructus Lycii, 16 g of Radix Puerariae Lobatae, 6 g of Radix Bupleuri, 12 g of Fructus Crataegi, 6 g of Semen Cassiae, 12 g of Fructus Gardeniae, 6 g of Fructus Hordei Germinatus, 12 g of Folium Nelumbinis, 4 g of Rhizoma Polygonati, 10 g of Endothelium Corneum Gigeriae Galli, 4 g of Flos Chrysanthemi, 8 g of Radix Angelicae Sinensis, 3 g of Radix et Rhizoma Rhei, 6 g of Ganoderma.

### Embodiment 2

The over-the-counter traditional Chinese medicine for weight loss comprises the following raw materials in parts by weight: 60 g of stir-fried yellow soybeans, 25 g of Gynostemma pentaphyllum, 15 g of Radix et Rhizoma ginseng, 8 g of Radix Astragali, 16 g of Fructus Lycii, 8 g of Radix Puerariae Lobatae, 12 g of Radix Bupleuri, 6 g of Fructus Crataegi, 12 g of Semen Cassiae, 6 g of Fructus Gardeniae, 12 g of Fructus Hordei Germinatus, 6 g of Folium Nelumbinis, 8 g of Rhizoma Polygonati, 10 g of Endothelium Corneum Gigeriae Galli, 8 g of Flos Chrysanthemi, 4 g of Radix Angelicae Sinensis, 6 g of Radix et Rhizoma Rhei, 3 g of Ganoderma.

### Embodiment 3

The over-the-counter traditional Chinese medicine for weight loss comprises the following raw materials in parts by weight: 45 g of stir-fried yellow soybeans, 38 g of Gynostemma pentaphyllum, 13 g of Radix et Rhizoma ginseng, 9 g of Radix Astragali, 12 g of Fructus Lycii, 12 g of Radix Puerariae Lobatae, 9 g of Radix Bupleuri, 9 g of Fructus Crataegi, 9 g of Semen Cassiae, 9 g of Fructus Gardeniae, 9 g of Fructus Hordei Germinatus, 9 g of Folium Nelumbinis, 6 g of Rhizoma Polygonati, 9 g of Endothelium Corneum Gigeriae Galli, 6 g of Flos Chrysanthemi, 6 g of Radix Angelicae Sinensis, 5 g of Radix et Rhizoma Rhei, 4 g of Ganoderma.

### Embodiment 4

The over-the-counter traditional Chinese medicine for weight loss comprises the following raw materials in parts by weight: 30 g of stir-fried yellow soybeans, 50 g of Gynostemma pentaphyllum, 10 g of Radix et Rhizoma ginseng, 10 g of Radix Astragali, 8 g of Fructus Lycii, 16 g of Radix Puerariae Lobatae, 6 g of Radix Bupleuri, 12 g of Fructus Crataegi, 6 g of Semen Cassiae, 9 g of Fructus Gardeniae, 9 g of Fructus Hordei Germinatus, 9 g of Folium Nelumbinis, 6 g of Rhizoma Polygonati, 9 g of Endothelium Corneum Gigeriae Galli, 6 g of Flos Chrysanthemi, 6 g of Radix Angelicae Sinensis, 5 g of Radix et Rhizoma Rhei, 4 g of Ganoderma.

### Embodiment 5

The over-the-counter traditional Chinese medicine for weight loss comprises the following raw materials in parts by weight: 60 g of stir-fried yellow soybeans, 25 g of Gynostemma pentaphyllum, 15 g of Radix et Rhizoma ginseng, 8 g of Radix Astragali, 16 g of Fructus Lycii, 8 g of Radix Puerariae Lobatae, 12 g of Radix Bupleuri, 6 g of Fructus Crataegi, 6 g of Semen Cassiae, 12 g of Fructus Gardeniae, 6 g of Fructus Hordei Germinatus, 12 g of Folium Nelumbinis, 4 g of Rhizoma Polygonati, 10 g of Endothelium Corneum Gigeriae Galli, 4 g of Flos Chrysanthemi, 8 g of Radix Angelicae Sinensis, 3 g of Radix et Rhizoma Rhei, 6 g of Ganoderma.

The invention is produced through the steps of drug selection, cleaning, drying, crushing, cell wall breaking, filling and packaging.
1. Animals: SD rats provided by the Experimental Animal Center of Sichuan Medical Science Academy (Sichuan Experimental Animal Management No. 24101101), weighing 220±10 g, with an equal number of males and females.
2. Feed: High-sugar combined with high-calorie feed (20% fat, 52% sucrose, 18% protein, and appropriate mixed salts, vitamins, fiber, etc.); ordinary feed.
3. Establishment of rat obesity animal model: animals were randomly divided into 2 groups, namely the control group was fed with ordinary feed and the obesity group was fed with high-calorie feed, with free access to food and water. The body weight was measured once a week before and after the experiment. After 2 weeks, when a significant difference in weight gain between the two groups was observed, the obesity model was considered established.
4. Weight loss experiment: the animals in the obesity group were randomly divided into an obesity control group, weight loss group 1, and weight loss group 2. The animals continued to be fed with high-calorie feed. The normal control group continued to be fed with ordinary feed, with animals having free access to food and water.

The tea extract was concentrated by using the water bath vacuum method to make 1 g of tea equivalent to 1ml of tea liquid. The weight loss group 1 was administered 0.5 g of tea per 100 g of body weight, and the weight loss group 2 was administered 1 g of tea per 100 g of body weight by using gastric gavage. The experiment lasted for 2 weeks.
5. Observing indicators: body weight, ratio of body fat weight to body weight (perirenal and retroperitoneal fat), triglyceride, total cholesterol, liver pathological indicators, and presence of side effects (diarrhea, decreased physical strength).
6. Results:

The test results of the weight loss effect of the product of the invention are as follows:

| | | Weight Gain | Ratio of Fat | | |
|---|---|---|---|---|---|
| | | before and after | to Body | Cholesterol | Triglyceride |
| | Number | the Experiment | Weight | mmol/L | mmol/L |
| Group | of Cases | (g) X = SD | X = SD | LX = SD | LX = SD |
| control group | 12 | 19.08 = 15.67 | 0.016 = 0.006 | 2.09 = 0.22 | 0.94 = 0.32 |
| obesity | 12 | 43.09 = 21.48 ^{a} | 0.035 = 0.017 ^{a} | 2.21 = 0.34 | 3.23 = 1.08 ^{a} |
| control group | | | | | |
| weight loss | 12 | 36.80 = 24.90 | 0.032 = 0.018 ^{a} | 2.33 = 0.28 | 2.30 = 0.94 ^{a.b} |
| group 1 | | | | | |
| weight loss | 12 | −3.20 = 22.70 ^{a.b.c} | 0.027 = 0.013 | 2.57 = 0.38 | 2.18 = 1.31 ^{a.b} |
| group 2 | | | | | |

^{a} significantly different from the control group;
^{b} significantly different from the obese control group;
^{c} significantly different from the weight loss group 1.

It can be seen from the table that after the experiment, the average weight gain of the obese control group was significantly higher than that of the normal control group (P<0.01); although the weight loss group also increased, there was no significant difference from the control group; the weight of the weight loss group 2 decreased significantly, which was significantly different from the other groups (P<0.01). Regarding the ratio of fat to body weight, both the obese control group and the weight loss group 1 were higher than the normal control group (P<0.05), while there was no significant difference between the weight loss group 2 and the normal control group. Regarding cholesterol, there were no significant differences between the groups. Regarding triglyceride, both weight-loss groups were lower than the obese control group (P<0.05), but they were lower than the normal control group (P<0.01). No obvious side effects were found on them during the experimental period. No abnormalities were found in liver pathological examination.
7. Conclusion: the product of the invention has the effects of losing weight and lowering blood lipids under experimental conditions.

### Experimental Projects and Test Results

The subjects were those with simple obesity and no other diseases. Standard adult weight (kg)=height (cm)−105, and those whose actual measured weight exceeds the standard weight by 10%- 30% are overweight. An experimental group and a control group are set up according to the random principle, with 20 cases in each group. The experimental group takes the product of the invention, and the control group takes ordinary tea. Both the experimental group and the control group control the intake of carbohydrates appropriately. Other conditions are the same for the two groups.

One course of treatment lasts for 5 days, and 6 courses of treatment are taken continuously for a total of 30 days. Observing indicators: 1. the indicators of weight loss effect include weight, waist circumference, abdominal circumference, hip circumference, and subcutaneous fat thickness measurement (upper arms, back); testing once before and after taking tea. 2. eight biochemical indicators: GPT, GOT, TB, TP, ALB, TC, TG, and GLU; testing once before and after taking the tea.

Experimental results: comparison of weight loss effects between the experimental group and the control group:
the weight of the experimental group decreased by 6.4 kg, the waist circumference decreased by 4.0 cm, the hip circumference decreased by 3.1 cm, the abdominal circumference decreased by 4.0 cm, the skin fat thickness: the upper arms decreased by 6.7 mm and the back by 3.8 mm.
the weight of the control group decreased by 1.4 kg, the waist circumference decreased by 1.20 cm, the hip circumference decreased by 1.0 cm, the abdominal circumference decreased by 2.6 cm, the skin fat thickness: the upper arms decreased by 3.4 mm and the back by 1.4 mm.

Except that the reduction in abdominal circumference was not significant, all other aspects showed significant differences in the two groups. After taking tea, the triglycerides in 8 cases dropped to the reference value range. There is no adverse reaction during taking the product of the invention.

After taking the product of the invention for 6 courses (30 days) in human trials, weight loss indicators such as weight, waist circumference, hip circumference, and skin fat thickness were significantly reduced. Compared with the control group, it has a significant difference and no obvious adverse reactions. Therefore, the product of the invention has obvious weight loss effect on human body.

From the first day of the weight loss, you can eat chicken, fish, eggs, meat, seafood, green vegetables, and other scientifically balanced foods every day, make sure to always eat well, stay full, and maintain a good body shape.

The above are only preferred specific embodiments of the invention, but the protection scope of the invention is not limited thereto. Any person familiar with the technical field can make equivalent substitutions or changes based on the technical schemes and inventive concepts of the invention within the technical scope disclosed by the invention, and they shall be covered by the protection scope of the invention.

1. An over-the-counter traditional Chinese medicine for weight loss comprises the following raw materials in parts by weight: 30˜60 g of stir-fried yellow soybeans, 25˜50 g of Gynostemma pentaphyllum, 10˜15 g of Radix et Rhizoma ginseng, 8˜10 g of Radix Astragali, 8˜16 g of Fructus Lycii, 8˜16 g of Radix Puerariae Lobatae, and 6˜12 g of Radix Bupleuri, 6˜12 g of Fructus Crataegi, 6˜12 g of Semen Cassiae, 6˜12 g of Fructus Gardeniae, 6˜12 g of Fructus Hordei Germinatus, 6˜12 g of Folium Nelumbinis, 4˜8 g of Rhizoma Polygonati, 8˜10 g of Endothelium Corneum Gigeriae Galli, 4˜8 g of Flos Chrysanthemi, 4˜8 g of Radix Angelicae Sinensis, 3˜6 g of Radix et Rhizoma Rhei, 3˜6 g of Ganoderma.

2. The over-the-counter traditional Chinese medicine for weight loss of claim 1, it comprises the following raw materials in parts by weight: 30 g of stir-fried yellow soybeans, 50 g of Gynostemma pentaphyllum, 10 g of Radix et Rhizoma ginseng, 10 g of Radix Astragali, 8 g of Fructus Lycii, 16 g of Radix Puerariae Lobatae, 6 g of Radix Bupleuri, 12 g of Fructus Crataegi, 6 g of Semen Cassiae, 12 g of Fructus Gardeniae, 6 g of Fructus Hordei Germinatus, 12 g of Folium Nelumbinis, 4 g of Rhizoma Polygonati, 10 g of Endothelium Corneum Gigeriae Galli, 4 g of Flos Chrysanthemi, 8 g of Radix Angelicae Sinensis, 3 g of Radix et Rhizoma Rhei, 6 g of Ganoderma.

3. The over-the-counter traditional Chinese medicine for weight loss of claim 1, it comprises the following raw materials in parts by weight: 60 g of stir-fried yellow soybeans, 25 g of Gynostemma pentaphyllum, 15 g of Radix et Rhizoma ginseng, 8 g of Radix Astragali, 16 g of Fructus Lycii, 8 g of Radix Puerariae Lobatae, 12 g of Radix Bupleuri, 6 g of Fructus Crataegi, 12 g of Semen Cassiae, 6 g of Fructus Gardeniae, 12 g of Fructus Hordei Germinatus, 6 g of Folium Nelumbinis, 8 g of Rhizoma Polygonati, 10 g of Endothelium Corneum Gigeriae Galli, 8 g of Flos Chrysanthemi, 4 g of Radix Angelicae Sinensis, 6 g of Radix et Rhizoma Rhei, 3 g of Ganoderma.

4. The over-the-counter traditional Chinese medicine for weight loss of claim 1, it comprises the following raw materials in parts by weight: 45 g of stir-fried yellow soybeans, 38 g of Gynostemma pentaphyllum, 13 g of Radix et Rhizoma ginseng, 9 g of Radix Astragali, 12 g of Fructus Lycii, 12 g of Radix Puerariae Lobatae, 9 g of Radix Bupleuri, 9 g of Fructus Crataegi, 9 g of Semen Cassiae, 9 g of Fructus Gardeniae, 9 g of Fructus Hordei Germinatus, 9 g of Folium Nelumbinis, 6 g of Rhizoma Polygonati, 9 g of Endothelium Corneum Gigeriae Galli, 6 g of Flos Chrysanthemi, 6 g of Radix Angelicae Sinensis, 5 g of Radix et Rhizoma Rhei, 4 g of Ganoderma.
